(19) United States
(12) Patent Application Publication
(10) Pub. No.: US 2023/0226155 A1
(43) Pub. Date: Jul. 20, 2023

(54) COMPOSITION FOR THE PREVENTION AND/OR TREATMENT OF THE RESPIRATORY TRACT INFECTIONS

(71) Applicant: TDC TECHNOLOGY DEDICATED TO CARE SRL, Milano (MI) (IT)

(72) Inventors: Martina BIANCARDI, Barcelona (ES); Biagio BIANCARDI, Napoli (NA) (IT)

(21) Appl. No.: 17/921,278

(22) PCT Filed: Apr. 26, 2021
(86) PCT No.: PCT/IB2021/053425

§ 371 Date: Oct. 25, 2022

(30) Foreign Application Priority Data
Apr. 29, 2020 (IT) 102020000009430

(51) Int. Cl. A61K 38/40; A61K 9/127; A61K 9/12; A61P 31/14; A61K 9/48

(52) U.S. Cl. CPC A61K 38/40; A61K 9/1276; A61K 9/12; A61P 31/14; A61K 9/48

(57) ABSTRACT

The invention relates to a composition comprising apolactoferrin, optionally in admixture with lactoferrin, carried via liposomes, as well as to products comprising such a composition, and to their use for the prevention and/or treatment of the conditions affecting the respiratory system, in particular the viral infections.

### STATE OF THE ART

The surface of the mucous membranes represents the first entry route to the human body for many pathogens, such as, for example, bacteria and viruses. Viruses, in particular the respiratory and intestinal viruses, are of significant epidemiological importance.

Examples of common human diseases caused by viruses include the common cold, flu, chickenpox, and herpes labialis. Among the possible diseases caused by viral infections there are also serious conditions such as AIDS, Ebola virus disease, avian flu and SARS. Furthermore, the viruses can cause damage to the epithelium that covers the internal body cavities communicating with the outside, such as the digestive system, respiratory system, urinary and genital systems. Such damage can promote the bacterial super-infection, which in turn can lead to clinical problems even more severe than the primary viral infection.

The Coronavirus strain called SARS-CoV-2 had never been identified before it was reported in Wuhan, China, in December 2019. The disease caused by such strain of Coronavirus has been called “COVID-19”.

The condition called COVID-19 shows itself, in particular, by respiratory, gastrointestinal and muscular alterations. The first cause of death by COVID-19 is due to a rapidly progressive interstitial pneumonia with concomitant alveolar inflammatory state.

In March 2020, following its rapid and wide spread, the World Health Organization has defined COVID-19 a pandemic.

It is therefore essential to identify substances that can be used to treat and/or prevent the viral infections, in particular those of the respiratory system, such as, for example, COVID-19.

Lactoferrin is a bilobed glycoprotein constituted by approximately 690 amino acids. Each lobe reversibly binds one ferric ion per molecule.

Each molecule of lactoferrin can bind a maximum of two ferric ions to itself and based on such saturation can exist in three distinct forms: apolactoferrin (iron-free), monoferric lactoferrin (bound to a single ferric ion) and hololactoferrin (which binds two ferric ions to itself).

Numerous uses of lactoferrin are known.

For example, in the Italian Patent number 0001394514, in the name of the present Applicant, a composition based on lactoferrin carried in nanolipids and its use for the topical treatment of the skin pathologies characterized by accumulation of heavy metals is described.

Furthermore, the European Patent Application EP3603621A1, in the name of the present Applicant, describes a product constituted by liposomes comprising lactoferrin and a component selected from hyaluronic acid or chitosan for its use in the prevention and/or treatment of the eye diseases, for example conjunctivitis, chalazion, stye, blepharoconjunctivitis and keratitis.

Uses of the iron-free form of lactoferrin, namely apolactoferrin, are also known. For example, JP2011093893 describes the use of apolactoferrin in the treatment of allergies. On the other hand, in the document JP2010229118 a product based on milk-derived proteins such as lactoferrin, apolactoferrin and enzymatic casein, which is capable of inhibiting lipase and thus advantageously used as adjuvant in the weight loss, is described.

WO2010005012 describes an apolactoferrin-based composition together with at least one additional ingredient selected from a glycated product-binding agent, an antioxidant substance and an antibacterial substance. The composition described can be used in the formulation of numerous products, for example food products, cosmetics, detergents, perfumes and others.

### AIMS OF THE INVENTION

Aim of the present invention is to provide a composition that is capable of preventing, alleviating and/or treating the respiratory tract infections, in particular viral infections, such as, for example, COVID-19.

A further aim of the present invention is to provide a composition for the treatment of the respiratory tract infections, when these have mild symptoms such as, for example, during the first stages of infection and/or in paucisymptomatic subjects.

Still another aim of the present invention is to provide a composition which is effective in the treatment and prevention of the infections, in particular viral infections, of the respiratory tract, and which does not have side effects or toxicity.

### BRIEF DESCRIPTION OF THE FIGURES

FIG. 1 is a graph showing the trend of rRT-PCR for SARS-COV-2 RNA

FIG. 2 is a graph showing the trend of the resolution of the clinical symptoms.

### DESCRIPTION OF THE INVENTION

The above aims, as well as other aims, are achieved by the object of the present invention, namely by a composition comprising apolactoferrin as active ingredient, included in liposomes. In fact, such composition can effectively be employed for the treatment of the infections, in particular viral infections, of the respiratory tract.

According to an advantageous aspect of the present invention, the composition can be used in the prevention and/or treatment of even serious viral infections such as, for example, SARS and COVID-19.

As mentioned above, the composition of the invention comprises apolactoferrin as active ingredient.

In the present description, the term “apolactoferrin” means lactoferrin in its iron-free form, i.e., not bound to ferric ions.

It has been surprisingly found that the apolactoferrin-based composition according to the present invention exhibits particular characteristics and enables remarkable results to be obtained in the treatment of the respiratory tract conditions caused by infections, in particular viral infections, also showing an unexpected effect with respect to the action of lactoferrin alone.

Not wishing to be bound to a specific scientific explanation, it has been hypothesized that apolactoferrin counteracts the viral infection by preventing the virus from entering the host cell, for example during the early stages of the infection. For what concerns the coronavirus infection, in particular the strain that causes COVID-19, it has been hypothesized that apolactoferrin interacts with particular adhesion proteins (spike proteins), which are present on the surface of the virus and mediate the entry into the host cell, performing its beneficial effects directly at the level of the viral capsid. Furthermore, apolactoferrin and lactoferrin have anti-inflammatory and immunomodulatory activities. The first activity depends on its ability to enter the host cell by endocytosis and be translocated at the nuclear level, leading to the expression of anti-inflammatory genes, also as an immunomodulatory agent it suppresses the expression of pro-inflammatory genes encoding interleukin 6 (IL6). Furthermore, it has been hypothesized that apolactoferrin, not being bound to ferric ions, can chelate the intracellular iron, rebalancing the intracellular REDOX state, thus reducing the inflammatory state resulting from the viral infection.

Furthermore, it has been found that the same advantages described above can be obtained when the composition of the invention comprises, in addition to apolactoferrin, also a certain amount of lactoferrin.

Therefore, according to an embodiment, the composition of the invention may also comprise lactoferrin, in addition to apolactoferrin, included in the liposomes.

In other words, according to further embodiments, the composition may comprise a mixture of apolactoferrin and lactoferrin as active ingredient, said mixture being included in liposomes.

In the present description, the term “lactoferrin” means lactoferrin in its form bound to at least one ferric ion. In other words, this term is intended to mean, for example, monoferric lactoferrin (bound to a single ferric ion).

When apolactoferrin is in admixture with lactoferrin, apolactoferrin is preferably in a higher amount than lactoferrin.

In different embodiments of the present invention, lactoferrin is in an amount between 0.001% and 50%, preferably between 0.01% and 25%, more preferably between 0.1% and 10% by weight to the total weight of apolactoferrin and lactoferrin.

The present invention also overcomes the limitations of the known art due to the ease of degradation and denaturation of the protein in its iron-free form, by providing a composition in which apolactoferrin is in the conformation not bound to ferric ions but, at the same time, is not substantially denatured or degraded. In fact, advantageously, in the composition of the present invention, apolactoferrin is included in liposomes.

In the present description, the term “liposome” is intended to denote a vesicle constituted by at least one lipid bilayer and an aqueous solution core encapsulated within the lipid bilayer. The lipids constituting the liposome bilayer (or liposome-forming lipids) may comprise mixtures composed primarily of phospholipids, such as phosphatidylcholine, and in lesser amounts of cholesterol. By way of example, lipids useful to constitute the lipid bilayer of the liposome are a mixture of cholesterol and soy lecithin, for example, comprising phosphatidylcholine (such as the commercial product Lipoid S75, or the commercial product Lipoid S80, currently sold by the company Lipoid Kosmetik AG, DE).

Other lipids useful for constituting the lipid bilayer of liposomes according to the present invention are the milk-derived phospholipids, in particular derived from the milk fat globule membrane (“MFGM”). Phospholipids derived from the milk fat globule membrane may contain, in addition to phosphatidylcholine, for example sphingomyelin and phosphatidylethanolamine (such as the commercial product “Milk Phospholipids”, currently marketed by the company A.C.E.F. S.p.A.). The liposome may also comprise additional non-lipidic compounds in its lipid bilayer, such as for example other organic compounds (such as tocopherol or lactose).

The liposomes can be produced by techniques per se known in the art such as, for example, extrusion and sonication.

According to embodiments of the present invention, the liposomes are produced by extrusion.

Advantageously, by varying parameters such as time, pressure and number of cycles, the extrusion can be used to produce liposomes having different dimensions, depending on the type of product to be obtained.

According to embodiments, the liposomes used in the composition of the invention comprise phospholipids derived from the membrane of milk fat globules. Advantageously, in this case, the liposomes are particularly gastro-resistant and, therefore, particularly adapted to be formulated in ingestible pharmaceutical forms such as, for example, capsules. However, the same liposomes con also be formulated in numerous other types of pharmaceutical formulations, for example in liquid formulations for sprays (e.g., nasal sprays) or aerosols.

According to embodiments, the liposomes used in the composition of the invention are constituted by phospholipids derived from the membrane of milk fat globules.

The apolactoferrin comprised in the liposomes according to the invention is preferably encapsulated within the liposomes, i.e., within the space bounded by the lipid bilayer, and more preferably is solubilized in the aqueous solution core that is typically within the liposome.

For reaching the lower airways the particles can be between 2000 and 4000 nm in dimension.

According to embodiments of the invention, the liposomes have an average diameter dimension between 25 nm and 1000 nm, preferably between 50 nm and 500 nm, more preferably between 70 nm and 250 nm. In embodiments, particularly preferred are the liposomes having an average diameter dimension between 80 nm and 200 nm.

Advantageously, when the liposomes have an average dimension of less than 1000 nm, preferably less than 500 nm, more preferably less than 300 nm, such liposomes are particularly stable and adapted to be formulated in the form of nasal sprays and aerosol formulations, as such dimensions allow the liposomes to reach the lower airways.

The dimension of the liposomes can be measured, as a z-average diameter, by using a particle analyzer by the dynamic light scattering (DLS) technique, e.g., by the Zetasizer Nano ZS instrument of Malvern Panalytical. Such measurements may be carried out after the liposomes have been appropriately diluted, e.g., by a 1:10 dilution with distilled water (e.g., Milli-Q).

The composition of the invention also has several advantages that make it particularly stable and effective for use in the treatment of the infections, in particular the viral infections. For example, apolactoferrin is stabilized thanks to its incorporation into the liposomes and is therefore protected from a possible denaturation. Furthermore, the composition of the invention is particularly adapted to be formulated in capsules, as well as in liquid formulations for sprays and/or aerosols, particularly when the liposome is formed of phospholipids derived from the membrane of milk fat globules.

According to embodiments, the liposomes according to the invention have negative Z potential. According to embodiments of the invention, the Z potential of the liposomes, measured in distilled water (e.g., Milli-Q, e.g., at a 1:10 dilution), may be in the range between −50 mV and −30 mV, preferably between −45 mV and −35 mV, more preferably between −42 and 38 mV. Such values can vary, even reaching positive values, i.e., higher than 0 mV, when the measurements are carried out by using a medium different from the buffer solution. The Z-potential values as described above can be measured by using a particle analyzer by the electrophoresis technique with Doppler laser, for example by the Zetasizer Nano ZS instrument from Malvern Panalytical.

According to embodiments of the invention, the liposomes have a polydispersion index between 0.100 and 0.400, preferably between 0.150 and 0.300, more preferably between 0.170 and 0.250.

The polydispersion index is a measure of the distribution uniformity of the molecular weights of the liposomes in a sample.

The polydispersion index values can be measured, e.g., by a 1:10 dilution with distilled water (e.g., Milli-Q), by using a particle analyzer, e.g., by the Zetasizer Nano ZS instrument from Malvern Panalytical.

According to embodiments, the liposomes may further incorporate pharmaceutically acceptable functional agents, such as for example cryoprotectants, osmolarity regulators, surfactants and buffers.

In particular, according to embodiments, the liposomes may incorporate one or more cryoprotectants. The cryoprotectants allow to maintain unaltered the chemical-physical characteristics of the liposomes, as well as the compositions comprising them. In fact, the cryoprotectants are useful, for example, if the liposomes are to be lyophilized for long-term storage. Cryoprotectants useful in the present invention may be, for example, mannitol, trehalose, cyclodextrin, and mixtures thereof, and preferably are mannitol, trehalose, and mixtures thereof.

Advantageously, the liposomes can be lyophilized for long-term storage. The liposomes can be lyophilized through techniques per se known in the art. Advantageously, the lyophilized liposomes in powder form are particularly stable and can be incorporated, preferably in powder form, inside capsules.

Optionally, the capsules may be opened and the lyophilized composition contained therein can be dissolved in warm water or other non-alcoholic beverages to ease their uptake by subjects with swallowing problems.

The lyophilized liposomes can, in addition, be used in the production of liquid compositions, such as sprays and aerosols.

In this case, the use of cryoprotectants is particularly advantageous, in particular in the liposome reconstruction phase, which corresponds to the phase of hydration of the liposome with water. The use of cryoprotectants ensures the integrity of the liposome once hydrated, by preventing the liposome breakage, not only during the lyophilization phase but also during the reconstruction phase.

In fact, during the preparation of the aqueous composition suitable to be used as a nasal spray or aerosol, the presence of cryoprotectants allows to avoid that liposomes are damaged or broken, and that their apolactoferrin-protecting function is compromised.

Furthermore, when the composition of the invention is in liquid form it may further comprise one or more components selected from: sorbitol, sodium chloride, EDTA, monobasic sodium phosphate, dibasic sodium phosphate, and water.

Osmolarity modulators useful in the present invention may be, for example, cationic electrolytes among which Na, K, Ca, Mg.

Further functional agents useful in the present invention, in particular for the preparation of the liposomes, are the surfactants. Advantageously, surfactants according to the present invention may be ionic or non-ionic surfactants, preferably non-ionic surfactants, such as, for example, polysorbate 80 (also named Tween® 80).

According to embodiments, the composition according to the invention further comprises pharmaceutically acceptable excipients.

As discussed above, the composition of the invention may be effectively employed for the prevention and/or treatment of the viral infections of the respiratory tract, for example COVID-19.

Advantageously, the composition of the invention may be employed for the treatment of the respiratory tract infections, when these have mild symptoms such as, for example, during the first stages of infection and/or in paucisymptomatic subjects.

Furthermore, in embodiments, the composition of the invention may be formulated in capsule, nasal spray or aerosol form. Advantageously, the liposomes formed from phospholipids derived from the membrane of milk fat globules can be used both in the capsules and in the liquid formulations, such as nasal spray and aerosol.

According to embodiments, capsules and nasal spray and/or aerosol can be used in the prevention and/or treatment of the viral infections of the respiratory tract and administered singly or in combination with each other.

Thus, another object of the present invention is a capsule comprising the composition of the invention, for the use in the prevention and/or treatment of the viral infections of the respiratory tract.

According to embodiments, the capsule comprises the composition in powder form, preferably lyophilized powder.

Still object of the present invention is a nasal spray or aerosol (i.e., a liquid composition adapted to be nebulized in nasal spray or aerosol form), for its use in the prevention and/or treatment of the viral infections of the respiratory tract.

Advantageously, the same composition, in liquid form, can be used to produce both nasal sprays and aerosols.

Preferably, if the composition is intended for the production of an aerosol, the composition is sterilized.

In embodiments, the nasal spray or aerosol comprises a mixture of apolactoferrin and lactoferrin as the active ingredient, included in liposomes, preferably encapsulated in liposomes.

Another object of the present invention is a combination of capsules with nasal spray and/or aerosol, comprising the composition according to the invention, for the prevention and/or treatment of the viral infections of the respiratory tract.

Capsules, nasal sprays and aerosols can be produced by techniques per se known in the art.

According to embodiments, each capsule includes 50 mg to 500 mg of apolactoferrin (optionally in admixture with lactoferrin), preferably between 75 mg and 250 mg, more preferably between 90 mg and 200 mg. In embodiments, each capsule includes about 100 mg of apolactoferrin or 100 mg of apolactoferrin and lactoferrin mixture).

According to embodiments, the liquid formulation suitable for the use as a spray contains apolactoferrin (optionally in admixture with lactoferrin), in a concentration between 0.1 mg/ml and 2. mg/ml, preferably between 0.5 mg/ml and 1.5 mg/ml, more preferably between 0.6 mg/ml and 1.0 mg/ml.

According to embodiments, the liquid formulation suitable for the use as an aerosol contains apolactoferrin (optionally in admixture with lactoferrin), in a concentration between 0.1 mg/ml and 2. mg/ml, preferably between 0.5 mg/ml and 1.5 mg/ml, more preferably between 0.6 mg/ml and 1.0 mg/ml.

When apolactoferrin is in admixture with lactoferrin, the amounts and concentrations described in the present invention refer to the sum of apolactoferrin and lactoferrin, except where explicitly stated otherwise.

The composition of the invention, as well as the products in which it is contained, in particular the liquid formulations for aerosols, may need to be sterile or substantially sterile, for example to meet regulatory requirements. For this purpose, it is possible to sterilize the composition of the invention, as well as the liposomes contained therein, by irradiation with γ-rays, for example at a dose of 25 kGy, or 10 kGy.

The invention could be even better understood thanks to the illustrative, non-limiting examples described in the following Experimental Section.

### EXPERIMENTAL SECTION

### Example 1

Liposome Preparation

For the preparation of the liposomes, milk-derived phospholipids, in particular derived from the milk fat globule membrane (“MFGM”), were used to produce the lipid bilayer, and apolactoferrin, or a mixture of apolactoferrin and lactoferrin, was used as active ingredient.

In particular, the commercial product “Fosfolipidi da latte” (currently marketed by the company A.C.E.F. S.p.A.) was used for the production of the lipid bilayer.

Mannitol and trehalose were also used as cryoprotectants.

The commercial product “Fosfolipidi da latte” has the following characteristics.

Composition:

TABLE 1

| Solids in milk | min. 95.0% |
|---|---|
| Phospholipid content | 16.0-21.0% |
| Phosphatidylethanolamine (PE) | 3.0-5.5% |
| Phosphatidylcholine (PC) | 4.0-5.5% |
| Phosphatidylinositol | 0.5-1.8% |
| Phosphatidylserine | 1.0-2.5% |
| Sphingomyelin | 3.5-5.0% |
| Ceramides* | 1.5-2.5% |
| Gangliosides | 0.3-0.6% |
| proteins | 50.0-60.0% |

*= mainly galactosyl- and glucosyl- ceramides

Chemical and Physical Analyses:

TABLE 2

| | |
|---|---|
| Consistency | Powder |
| Color | Whitish |
| humidity | max. 5% |
| Peroxide value | max. 3 meq/kg |

Microbiological Analyses:

TABLE 3

| | |
|---|---|
| Total plate count | max. 5,000 CFU/g |
| Yeasts and molds | <100 CFU/g |
| Coliforms | <10/g |
| Enterobacteriaceae | <10/g |
| Salmonella | negative/25 g |
| Listeria Monocytogenes | negative/25 g |

The liposomes were produced by the extrusion technique and lyophilized.

### Example 2

In Vitro Study of Liposome Stability Upon Digestion

1. Materials

Lyophilized liposomes comprising apolactoferrin (as obtained in Example 1); Milli-Q water; Distilled water; Micropipettes; 4 beakers; 37° C. bath; Zeta Sizer NanoZS.

2. Methods

2.1 Physical-Chemical Characterization

For this test, a solution of lyophilized liposomes with a concentration of 1 mg/ml was prepared.

1 mg (1.01 mg) of lyophilized liposomes was weighed and 10 ml of Milli-Q water were added.

A 1:10 dilution was then performed by using a P1000 micropipette. The dilution was carried out by using 100 μl of product and 900 μl of Milli-Q water, in a 2 ml Eppendorf tube.

This solution was transferred into a cuvette and the average diameter dimension (Zav), polydispersion index (PI) and Zeta potential (ZP) of the liposomes were analyzed in the Zeta Sizer instrument.

2.2. Preparation for Intestinal Release

Preparation of simulated gastric fluid (SGF) and simulated intestinal fluid (SIF):
SGF:
Weighing 2 g of NaCl and dissolving in about 100 ml of water.
Adding 7 ml of 2M HCl
Diluting up to 700 ml
Checking that the pH is close to 1.2 and if not, adding only HCL up to 1.2
Making up to 1 L
Once prepared and made up to volume with the 1 L volumetric flask, the solution is divided into two 250 ml flasks and one 500 ml flask to be kept in the refrigerator, which are labeled with the date of preparation.
In one of the two 250 ml flasks, 8 mg of pepsin is added, so as to obtain a pepsin concentration of 0.032 mg/ml.
SFI
Weighing 6.8 g of dipotassium phosphate and dissolving in 100 ml of Milli-Q water.
Adding 190 ml of a solution: 120-150 ml of 0.1 M NaOH, by adding 0.83 g of CaCl₂ and 1.66502 g of NaCl (0.1 M NaOH, 150 mM NaCl and 30 mM CaCl solution)
Making up to 900 ml and bringing the pH to 7.4
Adding 0.32 mg/ml of bile salts
Once prepared and made up to volume with the 1 L volumetric flask, the solution is divided into two 250 ml flasks and one 500 ml flask to be kept in the refrigerator, which are labeled with the date of preparation.
In one of the two 250 ml flasks, 0.032 mg/ml of pancreatin is added, in the other one not.

For this test, a bath was prepared that was heated at 37° C., then 4 solutions were prepared consisting of 24 ml of sample (1 mg/ml)+72 of SGF or SFI medium, with or without enzymes, then we will have 4 beakers with the following media:
SGF+Pepsin
SGF
SIF+Pancreatin
SIF

Once the appropriate bath temperature was reached, 24 ml (1 mg/ml) of lyophilized liposome sample were added to the 4 media. The beakers were set to agitation and once the sample was added to the beakers, the chronometer was started. 1 ml of solution was extracted from each of the beakers at the following times (min): 0, 15, 30, 60, 120. Each ml extracted was replaced with 1 ml of the corresponding medium. The samples extracted at different times, were passed through the Zeta Sizer NanoZS to measure the particle dimension, polydispersity index and Z potential.

3. Results

3.1 Physical-Chemical Characterization of Liposomes

The average results of the measurements made on the liposomes before being contacted with the different test media are set forth in the following table (Table 4):

TABLE 4

| Zav (nm) | PI | ZP (mV) |
|---|---|---|
| 192.9 ± 4.5 | 0.181 ± 0.02 | −40 ± 0.5 |

3.2 Behavior in the Gastric and Intestinal Medium

The average results obtained are set forth in the following tables (Tables 5, 6, 7 and 8):
SGF with Pepsin

TABLE 5

| Time (min) | Zav (nm) | PI | ZP (mV) |
|---|---|---|---|
| 0 | 260.5 ± 5.869 | 0.402 ± 0.021 | 17.1 ± 1.31 |
| 15 | 311.3 ± 11.60 | 0.326 ± 0.066 | 11.2 ± 0.451 |
| 30 | 266 ± 4.243 | 0.227 ± 0.017 | 11 ± 1.58 |
| 60 | 278.4 ± 37.34 | 0.360 ± 0.031 | 11.3 ± 0.569 |
| 120 | 236.8 ± 7.522 | 0.397 ± 0.025 | 10.9 ± 1.54 |

SGF

TABLE 6

| Time (min) | Zav (nm) | PI | ZP (mV) |
|---|---|---|---|
| 0 | 365.6 ± 60 | 0.412 ± 0.068 | 22.2 ± 0.265 |
| 15 | 530.3 ± 26.96 | 0.531 ± 0.016 | 22.6 ± 3.36 |
| 30 | 395.3 ± 96.17 | 0.519 ± 0.175 | 16.6 ± 0.495 |
| 60 | 348.3 ± 18.63 | 0.371 ± 0.014 | 20.2 ± 0.751 |
| 120 | 570.8 ± 26.50 | 0.508 ± 0.081 | 21.6 ± 1.31 |

SIF with Pancreatin

TABLE 7

| Time (min) | Zav (nm) | PI | ZP (mV) |
|---|---|---|---|
| 0 | 327.5 ± 12.87 | 0.455 ± 0.081 | −16.8 ± 0.551 |
| 15 | 318.2 ± 63.04 | 0.366 ± 0.055 | −17.7 ± 1.16 |
| 30 | 227.1 ± 2.828 | 0.401 ± 0.051 | −14.4 ± 4.45 |
| 60 | 255.6 ± 14.35 | 0.352 ± 0.053 | −12.3 ± 0.212 |
| 120 | 262.0 ± 22.35 | 0.358 ± 0.021 | −19.8 ± 1.86 |

SIF

TABLE 8

| Time (min) | Zav (nm) | PI | ZP (mV) |
|---|---|---|---|
| 0 | 964 ± 130.1 | 0.785 ± 0.120 | −18.9 ± 1.21 |
| 15 | 550.5 ± 89.43 | 0.532 ± 0.081 | −15.8 ± 0.896 |
| 30 | 612.9 ± 194 | 0.580 ± 0.077 | −20.9 ± 1.63 |
| 60 | 317.0 ± 24.08 | 0.369 ± 0.038 | −17.8 ± 1.40 |
| 120 | 326.9 ± 24.36 | 0.383 ± 0.042 | −17.3 ± 1.31 |

4. Conclusions

The contact of the liposomes with the intestinal and gastric delivery medium increases their dimension and PI. Especially in the case of the intestinal fluid.

The presence of enzymes (pepsin or pancreatin) promotes the maintenance of the average particle dimension.

The data obtained show that liposomes comprising apolactoferrin, obtained according to Example 1, in contact with SGF and SIF, while changing their characteristics, do not break down. The variation in the liposome characteristics is particularly reduced in the presence of enzymes (pepsin or pancreatin).

### Example 3

Use of a Combination of Food Supplements Both Based on Liposomal Apolactoferrin in Capsules and Nasal Spray in the Prevention and Treatment of COVID-19.—Protocol A

Study Design

A pilot study was designed in order to assess in detail the effects of using a combination of food supplements both based on liposomal apolactoferrin in capsules and nasal spray in three groups of patients: asymptomatic COVID-19 positive, paucisymptomatic COVID-19 positive and inpatients at risk for infection but COVID-19 negative.

It has been hypothesized that the simultaneous use of both forms would provide protection both at the systemic level and at the local level.

For this reason, the assumption of apolactoferrin (optionally in admixture with lactoferrin) in liposomes for oral administration is essential even in the phases of prevention i.e., in negative subjects but highly exposed to COVID-19.

Furthermore, it has been hypothesized that increasing the concentration of apolactoferrin in the circulation would ensure an increase in its concentration at the level of the respiratory mucous membranes, effectively assisting the antiviral activity carried out by apolactoferrin administered via the inhalation route.

Description of the Population Under Study

The population under study comprises subjects divided into 3 arms according to the scientific rationale of the study, plus a fourth control group, and are selected according to specific inclusion and exclusion criteria as well as in proportion to their respective age and gender groups in relation to the distribution of all positive cases (or, under certain conditions, even negative ones) by age and gender group registered in Italy by the Ministry of Health as of Mar. 31, 2020 (6:00 pm).

The first arm of COVID-19 positive patients is that of the asymptomatic ones, they start at T0 the treatment with liposomal apolactoferrin by oral administration twice a day and in spray 3 times a day, for 30 days. Sputum and venous sampling are collected at T0 at the same time as the delivery of the medical device that the patient uses at home.

The second arm is constituted by COVID-19 positive patients, paucisymptomatic patients having a body temperature of more than 37.5° C., cough, headache, asthenia, diarrhea, myasthenia, SPO2>93% or PaO2/FiO2>300 mmHg without oxygen inhalation. At T0 they start the treatment with liposomal apolactoferrin by oral administration two/three times a day (depending on body weight) and as a spray 3 times a day for 30 days.

The third arm is that of COVID-19 negative patients at risk and in active surveillance for previous contact with COVID-19 positive subjects.

At T0 they start the treatment with liposomal apolactoferrin by oral administration and as a spray twice a day for 30 days.

The fourth group, with the same characteristics as the previous one, is to be considered as a control group needed for carrying out all the possible comparisons foreseen by the protocol.

Main Objective

The primary objective of the study is the assessment of the clinical conditions of the enrolled subjects, following the intake and inhalation of liposomal apolactoferrin in the COVID-19 positive patients which are paucisymptomatic or asymptomatic or COVID-19 negative patients but at risk.

Secondary Objectives

The secondary objectives are going to assess the safety and tolerability of the medical device containing liposomal apolactoferrin and in addition the following parameters are analyzed from the venous sampling of the patients:
Complete blood count assessment at T0, T1 and T2
Assessment of the IL-1β, IL-6, IL-10, TNFα levels at T0, T1 and T2
at T1 and T2 assessment of the rate of absence of fever, remission of the respiratory symptoms, recovery rate of lung imaging
Recovery rate of the C-reactive protein (CRP)
Recovery rate of the biochemical criterion (CK)
Undetectable viral RNA rate (continuous twice)
Admission time

Inclusion Criteria
Subjects between the age of 18-20 years or above that are swab positive for COVID-19
subjects suffering from COVID-19 confirmed by oropharyngeal swab examination or by serological examination on plasma
Subjects at risk for COVID-19 infection in active surveillance for previous contact with COVID-19 positive subjects

Exclusion Criteria
Pregnant and lactating women.
Patients taking nitric oxide and nitrates
Patients with reported milk protein allergy
Patients with a previous history of bronchial hyperactivity
Patients suffering from pre-existing respiratory conditions

Methods

The liposomal apolactoferrin support at T0, after 15 days (T1) and after 30 days (T2) in the patients recruited in the following modes is assessed:

Group 1:

15 asymptomatic patients.

Assessment of the body temperature and any respiratory symptoms.

Treatment since T0 with:

1. liposomal apolactoferrin capsules (cps) of 200 mg (equal to 100 mg of apolactoferrin), 5 capsules a day of which 3 in the morning and 2 in the evening for 30 days. (total dosage 500 mg of apolactoferrin per day). To improve the patient compliance, the capsules can be opened and the contents dissolved in any (non-alcoholic) beverage at a temperature not exceeding 40° C.

2. nasal spray (mixture of liposomal apolactoferrin and lactoferrin): 2 sprays per nostril 3 times a day, inhaling deeply during the administration. It is recommended to thoroughly cleanse the nasal cavity before the administration.

Group 2:

15 paucisymptomatic patients in hospitalization but not in intensive care hospitalization.

Assessment of the body temperature, respiratory symptoms (rhinorrhea, cough), headache, conjunctivitis, myasthenia and diarrhea, possible lung imaging.

Treatment since T0 with:

1. liposomal apolactoferrin in 200 mg capsules (equivalent to 100 mg of apolactoferrin), 10 capsules per day for patients weighing less than or equal to 70 kg divided into 5 capsules in the morning and 5 capsules in the evening for 30 days for a total of 1 g of apolactoferrin/day; patients weighing more than 70 kg, 15 capsules per day divided into 3 administrations/day for 30 days for a total of 1.5 g of apolactoferrin/day. To improve the patient compliance, the capsules can be opened and the contents dissolved in any (non-alcoholic) beverage at a temperature not exceeding 40° C.

2. nasal spray: 2 sprays per nostril 3 times a day, inhaling deeply during the administration. It is recommended to thoroughly cleanse the nasal cavity before the administration.

Group 3:

15 patients at risk for previous contact with infected but COVID-19 negative patients in active surveillance.

Treatment since T0 with:

1. liposomal lactoferrin in cps of 200 mg (equal to 100 mg of lactoferrin), 2 capsules per day divided into 1 capsule in the morning and 1 capsule in the evening for 30 days for a total of 200 mg of lactoferrin/day. To improve the patient compliance, the same can be opened and the contents dissolved in any (non-alcoholic) beverage at a temperature not exceeding 40° C.

2. nasal spray (mixture of liposomal apolactoferrin and lactoferrin): 2 sprays per nostril 2 times a day, inhaling deeply during the administration. It is recommended to thoroughly cleanse the nasal cavity before the administration.

Group 4:

15 patients at risk for previous contact with infected but COVID-19 negative patients in active surveillance in the absence of treatment (control group).

The 15 patients enrolled in each of the aforementioned four groups have the same distribution in terms of age and sex, but diversified according to the group to which they belong; the distribution in question is proportional, as mentioned above, to the official distribution of the total number of the COVID19 positive cases detected by the Ministry of Health.

The following table (Table 9) reports this distribution in question for the four groups considered here:

TABLE 9

| DISTRIBUTION OF | | | | |
|---|---|---|---|---|
| POSITIVE CASES* | | | | |
| | PATIENT | | GROUPS | |
| AGE | PERCENTAGES | | NUMBER OF PATIENTS | |
| RANGE | males | females | males | females |
| >90 | — | 2.56 | — | 0 |
| 80-89 | 8.14 | 6.94 | 1 | 1 |
| 70-79 | 11.98 | 6.55 | 2 | 1 |
| 60-69 | 11.46 | 5.96 | 2 | 1 |
| 50-59 | 10.74 | 9.06 | 2 | 1 |
| 40-49 | 6.19 | 6.61 | 1 | 1 |
| 30-39 | 3.31 | 3.58 | 0 | 1 |
| 20-29 | 1.74 | 2.27 | 0 | 0 |
| TOTAL | 53.56 | 43.53 | 8 | 6 |

*Source: GEDI VISUAL—Coronavirus, the situation in Italy—update Mar. 31^{st}, 2020 at 6:00 p.m. Ministry of Health data.

During the follow-up on schedule of the patients (T1 and T2) starting from the first visit of enrollment (T0) a venous sampling is performed, subject to informed consent, for the assessment of the following parameters: complete blood count, PCR, CK, IL-6, IL-10, TNFα.

Statistical Analysis

Since this is a preliminary study, we did not proceed to the calculation of the sample dimension (power analysis) for lack of statistics suitable for the purpose for which we intend to enroll 60 patients: 15 in each of the four hypothesized groups. For what concerns the descriptive statistical analysis, central tendency, variability, symmetry and kurtosis are calculated. Graphs complete the visual description of the set of measured variables. Finally, the 2×2 contingency tables are constructed for all the crossings between the experimental and control variables and the related ODDS RATIO are calculated. From the inferential point of view, the statistical significance of the odds ratio is determined by means of Student's t and the related 95% confidence intervals are constructed; in addition to this, Pearson's χ2, Fisher's exact test, Mc Nemar's test and Yates' test are calculated. The experimental results are considered statistically significant if they have p-values≤0.05.

### Example 4

Use of a Combination of Food Supplements Both Based on Liposomal Apolactoferrin in Capsules and Nasal Spray in the Prevention and Treatment of COVID-19.—Protocol B

Clinical Trials

An interventional, prospective, randomized trial was carried out to assess the efficacy of a liposomal formulation of apolactoferrin in COVID-19 patients with mild to moderate disease and asymptomatic COVID-19 patients. Mild to moderate disease was defined based on less severe clinical symptoms, with no evidence of pneumonia and no need for admission to the intensive care unit (ICU).

The primary endpoint was the negative RNA conversion rate of SARS-COV-2 evidenced by real-time retro-transcriptional polymerase chain reaction (rRT-PCR).

The secondary endpoints provided for the identification of blood parameters altered by COVID-19 and, consequently, the definition of the target markers for the therapy and rate of disease remission, defined as resolution of symptoms and improvement of the blood parameters. The safety and tolerability of liposomal apolactoferrin for oral and intranasal use were also assessed.

Patients (Population Under Study)

The eligible patients were subjects older than 20 years with COVID-19 confirmed by rRT-PCR on nasopharyngeal swab and blood oxygen saturation (SPO2)>93% or Horowitz index (PaO2/FiO2)>300 mmHg. The patients had not received any other treatment against SARS-CoV-2. The exclusion criteria comprise: pregnancy and lactation, intake of nitric oxide and nitrates, known allergy to the milk proteins, positive medical history of bronchial hyperactivity or pre-existing respiratory conditions. COVID-19 patients admitted to the ICU were excluded.

A control group consisting of healthy volunteers with negative rRT-PCR on nasopharyngeal swab was also included in the study to match the aforementioned group. The matched-pair analysis examined the structural and clinical characteristics of the matched group. For ethical reasons, no placebo or liposome treatment arms were included.

All patients provided written informed consent after receiving a full explanation of the purposes and risks of the study. To be included, patients had to be able to understand the content of the informed consent form and sign it.

Apolactoferrin

For the clinical trial, capsules and nasal spray containing liposome-encapsulated apolactoferrin were used.

The apolactoferrin capsules contain 100 mg of liposome-encapsulated apolactoferrin (apo-Lf), while the nasal spray contains approximately 2.5 mg/ml of liposome-encapsulated apo-Lf. Apo-Lf, contained in both products, was verified by SDS-PAGE and silver nitrate staining and its purity was found to be about 95%. The iron saturation of apo-Lf was about 5%, according to the detection performed by optical spectroscopy at 468 nm with extinction coefficient of 0.54 (iron saturation of 100%, 1% solution).

Study Design

The COVID-19 patients were consecutively enrolled from Apr. 22, 2020 till Jun. 22, 2020 at “Tor Vergata” University Hospital, Pineta Grande Hospital in Caserta, and Villa dei Pini Nursing Home in Anzio (Rome). The dosage regimen of liposomal apolactoferrin for oral use provided for the administration of 1 g per day for 30 days (10 capsules per day) in addition to the same formulation administered intranasally 3 times per day.

The apolactoferrin capsules contain 100 mg of liposome-encapsulated apo-Lf, while the apolactoferrin nasal spray contains approximately 2.5 mg/ml of liposome-encapsulated apo-Lf. Apo-Lf, contained in both products, was verified by SDS-PAGE and silver nitrate staining and its purity was found to be about 95%. The iron saturation of apo-Lf was about 5%, according to the detection performed by optical spectroscopy at 468 nm with extinction coefficient of 0.54 (100% iron saturation, 1% solution).

The control group consisting of healthy volunteers received no treatment or placebo.

Endpoint Measurements

rRT-PCR was performed at T0, T1 (after 15 days) and T2 (after 30 days) to detect SARS-CoV-2 RNA in the population under study.

All participants (COVID-19 patients and control group) underwent the following laboratory tests: complete blood count and chemistry panel (liver and kidney function), iron panel, coagulation profile, IL-6, IL-10, TNFα, serum adrenomedullin levels. The blood samples from COVID-19 patients were taken at T0 and T2: the blood samples from the control group were taken at T0.

The body temperature measurement and assessment of related signs and symptoms were performed at T0, T1 and T2 in COVID-19 patients.

Statistical Analysis

Descriptive and inferential statistical analyses were carried out. The Kolmogorov-Smirnov test was used to verify the normal distribution of the blood parameters.

The blood parameters obtained at T0 in the COVID-19 group and the control group were compared by t-test. The data were then analyzed with a two-tailed significant p-value<=0.05.

All parameters obtained at T0 and T2 in the COVID-19 group were subsequently compared by paired t-test. Furthermore, the mean change between TO and T2 was also assessed by using paired t-test. The normally distributed data were then analyzed with a significant p-value<=0.05.

Results

Demographic Data

In total, 32 patients with COVID-19 infection confirmed by real-time retro-transcriptional polymerase chain reaction (rRT-PCR) were recruited to be included in the COVID-19 patient group for the participation in the study protocol. Twenty-two patients had mild to moderate symptoms, while 10 patients were asymptomatic. The mean age was 54.6±16.9 years. Fourteen patients were male and 18 were female. The most prevalent comorbidity was hypertension (28%), followed by cardiovascular conditions (15.6%) and dementia (12.5%).

Thirty-two healthy volunteers (mean age 52.8±15.5 years) with negative rRT-PCR for SARS-CoV-2 RNA were recruited into the control group to be matched with the COVID-19 group described above. The patient group and control group were homogeneous in terms of age and comorbidities. Clinical and demographic data for both groups are summarized in Table 10.

TABLE 10
Clinical and demographic data

| | | COVID-19 group | | CONTROL group | |
|---|---|---|---|---|---|
| | | Average | | Average | |
| Demographic data | | +/−SD | N (%) | +/−SD | N (%) |
| Age | | 54.56 +/− | | 52.83 +/− | |
| | | 16.86 | | 15.5 | |
| Sex | males | | 14 | | 13 |
| | | | (44%) | | (41%) |
| | females | | 18 | | 19 |
| | | | (56%) | | (59%) |
| Mild to | | | 22 | | |
| moderate | | | (68.7%) | | |
| patients | | | | | |
| Asympto- | | | 10 | | |
| matic | | | (31%) | | |
| patients | | | | | |
| Comor- | Hypertension | | 9 | | 7 |
| bidities | | | (28.1%) | | (21.9%) |
| | Dementia | | 4 | | 1 |
| | | | (12.5%) | | (3.1%) |
| | Cardiovascular | | 5 | | 5 |
| | pathologies | | (15.625%) | | (15.625) |
| | HCV | | 2 | | 0 |
| | infection | | (6.3%) | | |
| | Anemia | | 2 | | 2 |
| | | | (6.3%) | | (6.3%) |
| | Encephalo- | | 3 | | 0 |
| | pathy | | (9.4%) | | |
| | Adenomatous | | 2 | | 0 |
| | Polyposis Coli | | (6.3%) | | |

Primary Endpoint

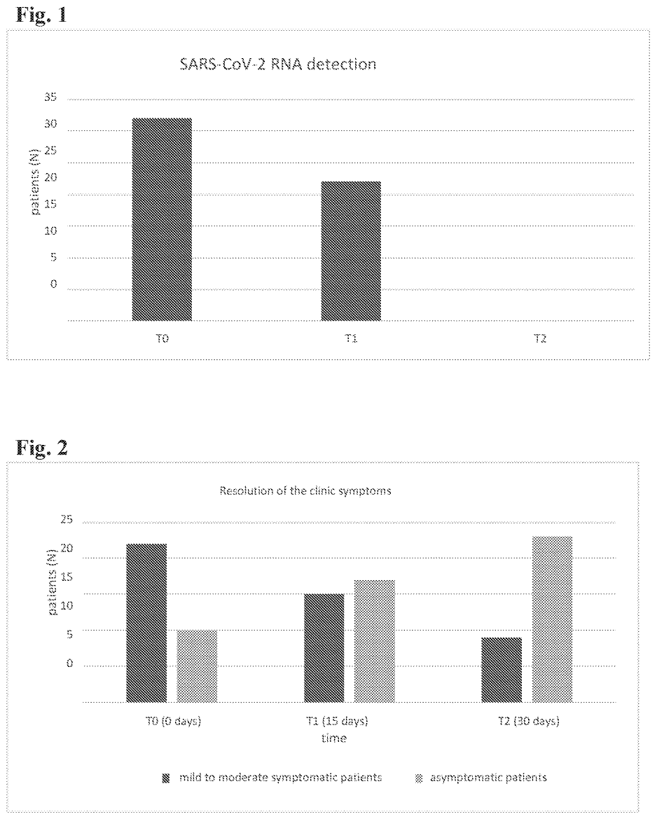

The real-time retro-transcriptional polymerase chain reaction (rRT-PCR) revealed a negative conversion of SARS-CoV-2 RNA in the nasopharyngeal swab in 10 patients (31.25%) at T1 and in all other patients at T2. All patients showed viral clearance at T2 (FIG. 1).

Secondary Endpoints

At T0, 22 patients were symptomatic and 10 patients were asymptomatic. The most frequent symptoms were fatigue (50%), arthralgia (37.5%) and cough (28%). At T1, 5 previously symptomatic patients became asymptomatic, for a total of 17 asymptomatic and 15 symptomatic patients. At T2, an additional 6 patients previously symptomatic at T1 became asymptomatic, for a total of 23 asymptomatic and 9 symptomatic patients. Among symptomatic individuals, the most frequent symptom was fatigue (21.9%). The clinical symptoms are summarized in FIG. 2.

When comparing the parameters of the COVID-19 group with the parameters of the control group at T0, there was a significant difference in terms of platelet count (p-value<0.0001), neutrophil count (p-value=0.04), monocyte count (p-value=0.006), D-dimer (<0.0001), aspartate aminotransferase (AST) (p-value=0.008), ferritin (p-value<0.0001), adrenomedullin (p-value<0.0001) and IL-6 (p-value<0.0001).

For what concerns the blood parameters of the COVID-19 group, the value of IL-6 showed a significant decrease between T2 and T0 (Δ_{T2-T0} −2.52±1.46, p-value=0.05). D-dimer also showed a significant decrease between T2 and T0 (Δ_{T2-T0} −392.56±142.71, p-value=0.01) and ferritin presented the same significant trend (Δ_{T2-T0} −90.63±48.49, p-value=0.04). However, the other values did not reach the statistical significance, however, an improvement in platelet count (T0: 239.63±83.05; T2: 243.70±65.5; ΔT_{T2-T0} 10.05±10.26) and a decrease in alanine transaminase (ALT) (T0: 29.36±22.7; T2: 23.52±12.34; Δ_{T2-T0} −7.32±4.36) and AST (T0: 24.36±9.80; T2: 22.64±8.33; Δ_{T2-T0} −2.68±2.52) was found. Adrenomedullin remained at the same level throughout the analyzed period (Δ_{T2-T0} −0.01±0.03). IL-10 levels increased between T0 (8.67±3.26) and T2 (11.42±6.05), without showing statistical significance (Δ_{T2-T0} 2.55±2.09). TNF-alpha decreased between T2 (25.97±21.74) and T0 (37.34±19.95), without showing statistical significance (Δ_{T2-T0} −12.92±8.81).

For what concerns the safety assessment, two patients (6.2%) experienced gastrointestinal problems related to taking apo-Lf at T2. The patients did not discontinue apo-Lf and the adverse event resolved spontaneously.

In this study, the focus was on the antiviral and immunomodulatory activity of apo-Lf as an effective therapeutic option against COVID-19.

Therefore, the role of apo-Lf was assessed in vivo, through a clinical trial, documenting its efficacy in promoting the viral clearance and gradual resolution of the symptoms in COVID-19 patients with mild to moderate disease and in asymptomatic COVID-19 patients.

In the study, apo-Lf induced early viral clearance after only 15 days from the treatment initiation in 31% of the patients and after 30 days of treatment in the rest of the patients.

In the study, some altered blood parameters suitable for the use as target markers of the treatment were identified. In fact, a statistically significant difference was found between the COVID-19 group and the control group in several blood parameters, including IL-6, D-dimer, ferritin and liver function parameters. In particular, IL-6, D-dimer and ferritin also showed a significant decrease after apo-Lf treatment, confirming as the most suitable target markers for the COVID-19 treatment.

Furthermore, we observed an increased platelet count after the apo-Lf treatment.

The reduction in ferritin levels during apo-Lf administration was found.

In the study, it was observed that the apo-Lf therapy reduced the transaminase levels, lowering the risk of liver injury in the COVID-19 patients.

In the study, adrenomedullin levels in the COVID-19 patients were assessed following the treatment with apo-Lf, noting that they remained constant between T2 and T0.

For what concerns the resolution of the clinical symptoms, a reduction in all the symptoms was observed, with the exception of fatigue which persisted in 21.9% of patients.

Regarding the safety of apo-Lf, gastrointestinal problems were reported in 2 patients, but these were occasional occurrences that did not lead to the discontinuation of the treatment.

In the analysis, formulations containing apolactoferrin encapsulated in liposomes were used for oral or nasal administration.

The statistical significance was reached in the crucial blood parameters related to the evolution of the disease and nevertheless an improving trend was observed in all the other markers analyzed.

It can also be stated that apo-Lf induced early negative RT-PCR conversion and rapid resolution of the clinical symptoms.

1. A method of treating respiratory tract infections in patients in need thereof with a composition comprising apolactoferrin as active ingredient included in liposomes, said method comprising
administering to said patients said composition.

2. The method according to claim 1, when said infections are viral infections.

3. The method according to claim 2, wherein said viral infection is COVID-19.

4. The method according to claim 1, wherein said composition comprises a mixture of apolactoferrin and lactoferrin as active ingredient, said mixture being included in said liposomes.

5. The method according to claim 4, wherein said apolactoferrin is in greater or equal quantity with respect to said lactoferrin.

6. The method according to claim 5, wherein said lactoferrin is between 0.001% and 50% by weight to the total weight of apolactoferrin and lactoferrin.

7. The method according to claim 1, wherein said liposomes comprise phospholipids derived from the membrane of milk fat globules.

8. The method according to claim 1, wherein said apolactoferrin is encapsulated within said liposomes.

9. The method according to claim 1, wherein said liposomes have an average diameter dimension between 25 nm and 1000 nm.

10. The method according to claim 1, wherein said liposomes include one or more cryoprotectants, selected from mannitol, trehalose, cyclodextrin, and mixtures thereof.

11. The method according to claim 1, wherein said liposomes are lyophilized.

12. The method according to claim 1, wherein said composition is in liquid or powder form.

13. A method of treating viral infections of respiratory tract in patients in need thereof with a capsule comprising said composition according to claim 1, said method comprising
administering to said patients said capsule comprising said composition.

14. The method according to claim 13, wherein said liposomes are lyophilized.

15. A method of treating viral infections of respiratory tract in patients in need thereof with a nasal spray or aerosol comprising said composition according to claim 1, said method comprising
administering to said patients the nasal spray or aerosol comprising said composition.

16. A method of treating viral infections of respiratory tract in patients in need thereof with a combination comprising a capsule comprising the composition according to claim 1 with a nasal spray or an aerosol comprising the composition according to claim 1, said method comprising
administering to said patients said combination comprising a capsule comprising the composition according to claim 1 with said nasal spray or said aerosol comprising the composition according to claim 1.

17. The method according to claim 6, wherein said lactoferrin is between 0.01% and 25% by weight to the total weight of apolactoferrin and lactoferrin.

18. The method according to claim 6, wherein said lactoferrin is between 0.1% and 10% by weight to the total weight of apolactoferrin and lactoferrin.

19. The method according to claim 9, wherein said liposomes have an average diameter dimension between 50 nm and 500 nm.

20. The method according to claim 9, wherein said liposomes have an average diameter dimension between 70 nm and 250 nm.
